(19) United States
(12) Patent Application Publication
(10) Pub. No.: US 2010/0201948 A1
(43) Pub. Date: Aug. 12, 2010

(54) APPARATUS FOR DETECTING RETINAL NEUROPATHY

(75) Inventors: Arash Sahraie, Aberdeenshire (GB); Paul Robert Kinnear, Aberdeen (GB); Augusto Azuora-Blanco, Aberdeen (GB)

Correspondence Address: WOODCOCK WASHBURN LLP, CIRA CENTRE, 12TH FLOOR, 2929 ARCH STREET, PHILADELPHIA, PA, 19104-2891, US

(73) Assignee: University Court of the University of Aberdeen, Aberdeen (GB)

(21) Appl. No.: 12/523,267

(22) PCT Filed: Jan. 15, 2008
(86) PCT No.: PCT/GB2008/000123

§ 371 Date: Apr. 27, 2010

(30) Foreign Application Priority Data
Jan. 15, 2007 (GB) 0700770.1

(51) Int. Cl. A61B 3/02

(52) U.S. Cl. 351/239; 351/246

(57) ABSTRACT

Screening apparatus includes a display for presenting a target to a participant; means for locating the participant's eye a predetermined distance from the display; means for animating the target so as to vary the speed of the target discontinuously; and evaluation means for determining the participant's ability to perceive the change in the target's speed.

This invention relates to an apparatus and method for detecting retinal neuropathy and, in particular, for determining whether a patient is able to detect a change in speed of a target.

It is known that retinal neuropathies such as glaucoma, diabetes and optic neuritis effect one's ability to perceive motion. In the past, tests to detect such retinal neuropathies have involved measuring a participant's reaction to a moving target.

The inventors of the present invention have come to realise however that the accuracy and reliability of such tests can be significantly improved by judging a participant's ability to perceive a change in a target's speed.

According to a first aspect of the invention, a screening apparatus is provided, the screening apparatus comprising a display for presenting a target to a participant; means for locating a participant's eye a predetermined distance from the display; means for, animating the target so as to vary the speed of the target discontinuously; and evaluation means for determining the participant's ability to perceive the change in the target's speed.

A discontinuous change in speed of the target occurs at a set point in space and therefore a participant's reaction to this can be more accurately evaluated than if the speed is gradually changed.

Catch trials wherein the speed of the target is not changed may be used to determine if the patient is reacting to an imagined change.

The speed of the target may be changed from a primary speed to a secondary speed wherein the primary and secondary speeds range between 0.5 and 20 degrees per second. All speeds are specified as measured on the display, relative to the eye of a participant, for the sake of convenience. For the tests to be meaningful, it is important that the participant's eye be located a set distance from the screen and means to do so, such as a chin rest, are provided.

Specifically, the primary speed may be about 1 degree per second and the secondary speed may be about 3 degrees per second.

The secondary speed may be incrementally increased until an indication is received from the participant that the change in speed was perceived. The indication may be a reaction from the participant such as the pushing of a button connected to a computer which logs the participant's response.

In addition, the secondary speed may be incrementally decreased until an indication is received from the participant that the change in speed is no longer perceived. The indication of lack of perception may be a lack of reaction from the participant.

By changing the increments by which the secondary speed is increased or decreased, the smallest change in speed a particular participant is able to perceive (of the increments used) can be identified. It has been found that there is a correlation between the size of the increment the participant is able to perceive and the likelihood that that participant is suffering from retinal neuropathy.

Successive incremental changes in speed may describe a linear relationship or a logarithmic relationship. It has been found that using a logarithmic relationship achieves the same result in a shorter time frame as using a linear relationship, and is therefore to be preferred.

It has further been found that the test is most effective where the target is presented to the participant at an offset, relative to a central region of the screen on which the participant's gaze may be fixed during use, of between −20 and +20 degrees and, in particular, of either −10 degrees or +10 degrees relative to the central region.

To ensure that the horizontal position of the target does not influence the tests, the position of the target is randomly alternated between a positive offset and a negative offset. As used herein relative terms such as “horizontal” are used with reference to a participant's perception during use of the apparatus.

It has further been found that optimal results are achieved where the target is presented to the participant at the primary and secondary speeds for time periods between 250 and 600 milliseconds (ms). Any period less than 250 milliseconds will affect the accuracy of the categorisation. Periods longer than 600 ms are impractical, although periods of up to 1600 ms may be used in practice runs to illustrate the workings of the tests.

The primary duration may be between 500 and 600 ms and the secondary duration may be between 250 and 400 ms.

It is to be realised that the terms “animation”, “speed” and “movement” are used to describe properties of the target. However what is important is that the participant perceive these properties and it is not necessary that the target be a physical object. Instead a computer screen simulating movement of a target, or other means of presenting the illusion of movement, may be used.

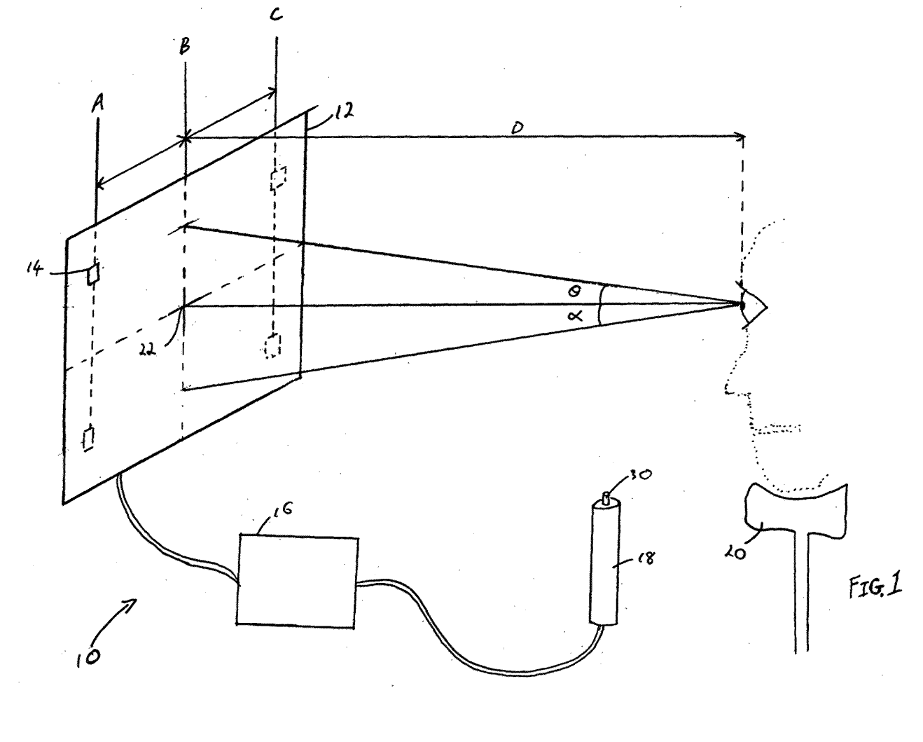

A preferred embodiment of the invention will now be described with reference to FIG. 1 which is a schematic representation of a screening apparatus according to the invention.

FIG. 1 illustrates a screening apparatus 10 which comprises a display 12 on which a target 14 is presented, a computer 16 connected to the display 12, a participant interface 18 and a chin rest 20. In the embodiment shown, the display 12 is a computer monitor controlled by the computer 16 having a central region 22. The chin rest 20 and the display 12 are located so that a participant's eye is positioned a set distance D away from the display 12.

It is to be realised that the choice of D will depend on the size of the display 12. What is important is that the target be presented to the participant at a vertical angle θ above, or α below, the central region 22 of about 10°, measured at the participant's eye. It has been shown that it is this region of the participant's field of vision (when focussing on the central region) that is most affected by retinal neuropathies, and hence the test is most effective when the target is presented in this region at a positive or negative offset of 10°.

During a test the target 14 moves along the display 12 at the horizontal line defined by the angles θ or α. In the tests described herein, the target was randomly presented either above or below the central region 22.

The target 14 is presented to a participant as moving along the screen 12 at a first, primary speed between the horizontal locations A and B. In certain runs of the test, the speed of the target moving across the display 12 is changed at location B, corresponding to the central region 22 and the target 14 moves at a secondary speed, different to the primary speed, between horizontal locations B and C.

As the target 14 is controlled by the computer 16, the perceived speed of the target 14 at position B can be instantaneously changed from the primary to the secondary speeds.

However in some runs of the test, the speed of the target is not altered at location B and the secondary speed is then the same as the primary speed. Such runs are known as “catch trials” and are intended to identify a participant unable to perceive a change in speed who nonetheless attempts to guess at the result.

The participant interface 18 includes a button 30 which, when depressed, sends a signal to the computer 16 which registers the event. During a test the participant will depress the button 30 when she believes that she has perceived a change in the speed of the target 14. The computer registers the depression of the button 30 and correlates this with the manner in which the movement of the target 14 is controlled.

Each time the participant has to decide whether a change in speed of the target 14 occurred is termed a “trial”. To determine the ability of the apparatus to categorise participants according to their ability to perceive a change in speed, two tests, each comprising a plurality of participants and, for each participant, a plurality of trials were conducted. The participants are either healthy (“normal”) or known to suffer from the retinal neuropathy, glaucoma.

In both tests, the primary speed is set at 1 degrees/second (°/s) and the secondary speed is initially set at 3°/s. If the participant is able to perceive the change in speed, the button 30 is depressed and the computer 16 registers this event. In the next trial, the secondary speed is then decreased by the first increment.

If the participant does not depress the button 30 within a period of 3 seconds of being shown the change in speed of the target, the computer 16 registers this as an indication that the participant did not perceive the change in speed. In this case, the secondary speed is increased by the first increment in the next trial.

For each successive trial for a given participant, the secondary speed is then increased or decreased by a predetermined amount (i.e. incremented or decremented by the first increment) until the participant's response changes. A change from increasing the speed to decreasing (or from decreasing to increasing) is termed a “reversal” and the computer 16 records the secondary speed at which this occurs.

On a reversal, the increment is reduced by a set amount and the process repeats with the new increment. Different tests using different relationships between successive increments.

The secondary speed is not increased above a predefined maximum and if the secondary speed reaches this maximum, it will be automatically decreased in the next trial, this counting as a reversal. Each participant is subjected to a plurality of test blocks, each test block comprising a predefined number of reversals.

A participant is then categorised according to the magnitude of the secondary speeds of the reversals of their test blocks.

Two successful tests were carried out.

In the first test, each participant underwent three test blocks, each test block comprising 10 reversals. The time period for which the target is presented to the participant at the primary speed is the primary duration and similarly, the duration at which the target is presented to the participant at the secondary speed is the secondary duration. In this test, the primary duration varied between 500 and 600 ms and the secondary duration varied between 250 and 400 ms.

The secondary speed had an initial speed of 3°/s and the successive increments used when a reversal occurred are set out (in °/s) in Table 1.

TABLE 1

| 1 | 2 | 3 | 4 | 5 | 6 | 7 | 8 | 9 | 10 |
|---|---|---|---|---|---|---|---|---|---|
| 0.2036 | 0.1866 | 0.17 | 0.1527 | 0.1357 | 0.1187 | 0.1018 | 0.0848 | 0.0679 | 0.0509 |

A maximum speed of 4°/sec was set for the secondary speed. If a participant did not notice a difference between the primary and secondary speeds where the secondary speed was 4°/sec, the test continued as if a reversal had occurred (i.e. the secondary speed was then decreased by the next increment).

As previously mentioned, the target is randomly presented either above (an upper score) or below (a lower score) the central region. For each trial block, the last six reversals are averaged, thereby deriving a number representative of the patient's upper score and a number representative of their lower score. There are three trial batches and therefore three “upper” results and three “lower” results.

Then, one of the following two methods, both of which are successful, are used to distinguish healthy individuals from those with retinal neuropathies. One method is to look at the largest scores in the upper and lower results and then choose the largest of the two. Alternatively, we can obtain the medians for the upper and the lower results and average these.

A second test was performed which was similar to the first test, but used the changes in the increment shown in Table 2.

TABLE 2

| 1 | 2 | 3 | 4 | 5 |
|---|---|---|---|---|
| 0.270 | 0.153 | 0.102 | 0.051 | 0.034 |

There are a fewer number of changes to the increment in the second test. Successive changes describe a logarithmic relationship. The results in this test were evaluated in the same manner as the results in the first test. The results of both tests are set out in Table 3.

TABLE 3
Using the mean of the upper and lower trials
Normal Participants

| | | |
|---|---|---|
| Test 1 | 2.59 | t(61) = 0.35, p = 0.73 |
| Test 2 | 2.56 | |

Glaucoma Participants

| | | |
|---|---|---|
| Test 1 | 3.04 | t(55) = 0.31, p = 0.76 |
| Test 2 | 3.00 | |

Using larger of the upper or lower trials
Normal Participants

| | | |
|---|---|---|
| Test 1 | 2.80 | t(61) = 0.72, p = 0.47 |
| Test 2 | 2.71 | |

Glaucoma Participants

| | | |
|---|---|---|
| Test 1 | 3.39 | t(55) = 0.33, p = 0.75 |
| Test 2 | 3.34 | |

It was found that there was no appreciable difference between the results of the first and second tests in differentiating between healthy patients and those with a retinal neuropathy such as glaucoma. Therefore the results were combined (see Table 4—only those results derived from the larger of the upper and lower trials is shown, although the mean value could equally have been chosen).

Using larger of the upper or lower trials

| | |
|---|---|
| Normal Participants | 2.75 |
| Glaucoma Participants | 3.36 |

The statistical difference between the two groups being t(118)=−6.92, p<0.000.

As these results show, both of the tests are successful at differentiating between participants who suffer from glaucoma and those who are healthy. A person may therefore be subjected to a process comprising either the first or the second test described above (or a combination of both) and the likelihood that they are suffering from glaucoma may be assessed with a relatively high degree of accuracy.

1.-42. (canceled)

43. Screening apparatus comprising:
a display for presenting a target to a participant;
means for locating the participant's eye a predetermined distance from the display;
means for animating the target so as to vary the speed of the target discontinuously; and
evaluation means for determining the participant's ability to perceive the change in the target's speed.

44. Apparatus according to claim 43 wherein the animating means changes the speed of the target from a primary speed to a secondary speed.

45. Apparatus according to claim 44 wherein the primary and secondary speeds range between 0.5 and 20 degrees per second, as measured relative to the location of a participant's eye during use.

46. Apparatus according to claim 44 wherein the evaluation means increases the secondary speed by an increment in successive trials until an indication is received from the participant that the change in speed was perceived, said indication being an indication of a first type.

47. Apparatus according to claim 44 wherein the evaluation means decreases the secondary speed by an increment until an indication is received from the participant that the change in speed is no longer perceived, said indication being an indication of a second type.

48. Apparatus according to claim 47 wherein a size of the increment is changed when the type of indication changes for successive trials.

49. Apparatus according to claim 48 wherein successive changes to the size of the increment describe a linear relationship or a logarithmic relationship.

50. Apparatus according to claim 43 wherein the display means has a central region about which a participant's vision is fixed during use and wherein the display means presents the target to the participant at an offset of between −20 degrees and +20 degrees relative to the central region, as measured relative to the location of a participant's eye during use.

51. Apparatus according to claim 50 wherein the eccentricity of the target is varied from a positive offset to a negative offset.

52. Apparatus according to claim 44 wherein the display presents the target to a participant for a primary duration at the primary speed and for a secondary duration at the secondary speed.

53. A method of categorizing a participant, the method comprising the steps of:
locating a participant's eye a predetermined distance from a display on which a target is displayed;
animating the target so as to change the speed of the target discontinuously;
determining the participant's ability to perceive the change in the target's speed; and
categorizing the participant based on their ability to perceive the change in the target's speed.

54. A method according to claim 53 wherein the speed of the target is changed from a primary speed to a secondary speed.

55. A method according to claim 54 wherein the primary and secondary speeds range between 0.5 and 20 degrees per second, as measured relative to the location of a participant's eye during use.

56. A method according to claim 54 wherein the secondary speed is increased by an increment in successive trials until an indication is received from the participant that the change in speed was perceived, said indication being an indication of a first type.

57. A method according to claim 54 wherein the secondary speed is decreased by an increment until an indication is received from the participant that the change in speed is no longer perceived, said indication being an indication of a second type.

58. A method according to claim 56 wherein a size of the increment is changed when the type of indication changes for successive trials.

59. A method according to claim 58 wherein successive changes to the size of the increment describe a linear relationship or a logarithmic relationship.

60. A method according to claim 53 wherein the display means has a central region about which a participant's vision is fixed during use and the target is presented to the participant at an offset of between −20 degrees and +20 degrees relative to the central region.

61. A method according to claim 54 comprising the further step of presenting the target to a participant for a primary duration at the primary speed and for a secondary duration at the secondary speed.

62. A method of detecting retinal neuropathy comprising the steps of:
locating a participant's eye a predetermined distance from a display on which a target is displayed;
animating the target so as to change the speed of the target discontinuously;
determining the participant's ability to perceive the change in the target's speed;
categorizing the participant based on their ability to perceive the change in the target's speed; and
using said categorization as an indication of the presence of retinal neuropathy.
